(19) United States
(12) Patent Application Publication
(10) Pub. No.: US 2020/0367762 A1
(43) Pub. Date: Nov. 26, 2020

(54) Methods and Systems for Patient Monitoring

(71) Applicant: Arthur Wallace, San Rafael, CA (US)

(72) Inventor: Arthur Wallace, San Rafael, CA (US)

(21) Appl. No.: 16/987,285

(22) Filed: Aug. 6, 2020

Related U.S. Application Data
(63) Continuation-in-part of application No. PCT/US2019/017065, filed on Feb. 7, 2019.
(60) Provisional application No. 62/627,632, filed on Feb. 7, 2018.

(51) Int. Cl. A61B 5/0205; A61B 5/145; A61B 5/00; A61B 5/113; A61B 5/103; A61B 5/026; A61B 5/11

(52) U.S. Cl. CPC A61B 5/0205; A61B 5/145; A61B 5/7257; A61B 5/1135; A61B 5/1115; A61B 5/0077; A61B 5/1034; A61B 5/0261; A61B 5/747; A61B 5/746

(57) ABSTRACT

This document describes methods and materials for improving patient monitoring. For example, this document describes methods and devices for reducing the risk of respiratory arrest, cardiac arrest, brain damage, and/or death by performing audiovisual detection of respiration, heart rate, oxygen saturation, perfusion, blood pressure, and/or motion.

### CROSS-REFERENCE TO RELATED APPLICATIONS

This application is a continuation-in-part application of and claims priority to U.S. International Application Serial No. PCT/US2019/017065, filed on Feb. 7, 2019, which claims the benefit of U.S. Provisional Application Ser. No. 62/627,632, filed Feb. 7, 2018. The disclosure of the prior applications is considered part of (and is incorporated by reference in) the disclosure of this application.

### BACKGROUND

### 1. Technical Field

This document relates to improving patient monitoring, for example, by using audiovisual information to monitor a patient.

### 2. Background Information

Patients in the hospital receive narcotics (morphine, dilaudid, fentanyl) for pain relief after surgery. These narcotics are delivered either through patient controlled analgesia machines (PCA) or by injection from nurses. All narcotics (μ agonists-opioid drugs that bind to μ receptors in the brain) tend to cause respiratory depression and can lead to respiratory arrest. The dose response relationship in narcotic-naïve patients has a ten-fold variability and dose response variability can be much greater (e.g., 1000 fold) in patients with prior exposure to narcotics. Patients who have an overdose of narcotics can have respiratory depression and can suffer cardiac arrest with brain damage and or death. The series of events including narcotic pain medication, respiratory arrest, and death is commonly known as the “found-dead-in-bed” phenomena.

Many approaches have been attempted to prevent respiratory arrest and death associated with narcotic administration including use of patient controlled analgesia (PCA) machines, which use a computer to limit the dose and require patient feedback prior to re-dosing. Patients on PCA narcotics routinely desaturate (e.g., 10%) despite oxygen supplementation and have apneic (not breathing) episodes (e.g., 3%). Additionally, pulse oximetry can be used to detect desaturation, however, patients often do not like to wear finger probes and take them off due to the fact that the probes limit use of the hands. Reflectance pulse oximetry can be used but there is a high incidence of patients taking off the pulse oximetry probe. Supplemental oxygen can reduce desaturation and reduces the risk of death and permanent brain damage after respiratory arrest, but, again, patients tend to remove the nasal cannula. Capnography, the measurement of carbon dioxide production, can be used to detect breathing but has a high failure rate secondary to patients removing the nasal cannula or breathing through their mouths. Despite the use of pulse oximetry, capnography, and supplemental oxygen, patients stop breathing after narcotic analgesia (pain relief) and suffer cardiac arrest and death. Patients with obstructive sleep apnea (OSA) have an even higher risk of respiratory arrest with narcotic pain relief.

Monitoring of cardiovascular and respiratory function in patients allows the detection of alterations in cardiovascular function. Monitoring of heart rate, respiration, pulse oximetry, and perfusion can detect events prior to clinical deterioration that can result in respiratory and cardiac arrest. Successful recovery from cardiac arrest can be poor and can be improved by rapid detection and treatment. Continuous heart rate (HR) monitors in patients at present utilized electrocardiograms that required adhesive electrodes on the skin. Measurement of respiratory rate (RR) requires either electrodes on the skin and impedance based measurements or sound detection of respiration or capnography from exhaled gases. Pulse oximetry (SaO2) requires a sensor attached to the patient on the finger, ear lobe, nose, or surface of the patient's skin. Cardiac and respiratory monitoring requires sensors, such as tubes for sensing gas exchange, to be attached to the patient's skin, and/or appendages. These sensors, wires, and/or tubes limit patient mobility and require skilled placement. Patients in hospitals may remove the sensors defeating the monitoring. Patients at home or in nursing homes often resist or defeat continuous monitoring with sensors that require attachment.

### SUMMARY

This document describes methods and materials for improving patient monitoring. For example, this document describes methods and devices for reducing the risk of respiratory arrest, cardiac arrest, brain damage, death, and/or other complications by performing audiovisual detection of respiration, heart rate, oxygen saturation, and perfusion.

In one aspect, this disclosure is directed to a monitoring device. The monitoring system can include a RGB (Red, Green, Blue) Video Camera, an Infrared Video Camera, a Stereoscopic Camera, a 3 Dimensional (3D) Depth Camera, a Microphone array, and software to analyze the video signals to provide measurement of respiratory rate, heart rate, pulse oximetry yielding oxygen saturation, perfusion, blood pressure, apnea, obstructive respiration, dis-coordinate breathing, vomiting, aspiration, out of bed alarm, patient call and communication system, and video conferencing for nurse call. The device can be used in hospitals including intensive care unit, step down units, and standard hospital beds. The device can also be used in nursing homes, assisted living, or in private homes. The device can be tailored for use in the elderly at home or for infants in NICUs, hospital beds, or at home as an apnea monitor, such as to prevent sudden infant death, or monitor other chronic medical conditions. The device can also be tailored to identify infants or children left in cars to help avoid “hot-car-deaths.”

Particular embodiments of the subject matter described in this document may be implemented to realize one or more of the following potential advantages.

First, embodiments of the monitoring device described herein can provide a non-invasive monitoring system. Second, the monitoring device can provide detection of respiratory rate, apneic events, and respiratory or cardiac arrest without any sensor touching a patient. Third, the monitoring device can provide remote pulse oximetry of the patient and can calculate oxygen saturation without any patient contact. Fourth, the monitoring device can measure perfusion and blood pressure without any patient contact. Fifth, the monitoring device can detect when the patient is out of bed. Sixth, the monitoring device can remotely call a nurse, or other person. Seventh, the monitoring device can provide selective, focused, detection of speech of the patient and not others in the room. Eighth, the monitoring device can detect snoring and other respiratory sounds. Ninth, the monitoring device can trigger remote video surveillance. Tenth, the monitoring device can trigger an alert or alarm. Eleventh, the monitoring device can control equipment in a room with the patient.

Therefore, the methods and systems provided herein allow remote, non-contact, monitoring of respiration, heart rate, pulse oximetry, perfusion, blood pressure, coordination of breathing, obstructive breathing, apnea, vomiting, mobility, location in bed for bed alarms, and facial and body symmetry for assessment of stroke without any physical contact with the patient. The device can include smart alarms to reduce or eliminate false alarms. The device can have predict impending respiratory and cardiac compromise, which may lead to respiratory and cardiac arrest.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

Details of one or more embodiments are set forth in the accompanying drawings and the description below. Other features, objects, and advantages will be apparent from the description and drawings, and from the claims.

### DESCRIPTION OF DRAWINGS

FIG. 1 is a perspective view of a patient in a room, in accordance with some embodiments provided herein.

FIG. 2 is a perspective view of the monitoring device of FIG. 1, in accordance with some embodiments provided herein.

FIG. 3 is a schematic view of a system of the monitoring device of FIG. 2, in accordance with some embodiments provided herein.

Like reference numbers represent corresponding parts throughout.

### DETAILED DESCRIPTION

This document describes methods and materials for improving patient monitoring of health status. For example, this document describes methods and devices for reducing the risk of respiratory arrest, cardiac arrest, brain damage, falls, and/or death by performing audiovisual detection of respiration and/or motion.

High doses of narcotics can cause respiratory arrest and death. In order to prevent death, sensors can be attached to a patient in order to monitor respiration, among other things. However, low patient compliance can lead to devices being removed.

Methods and devices provided herein can provide patient monitoring to reduce the risk of death without contact with the patient, resolving the issue of non-compliance by patients.

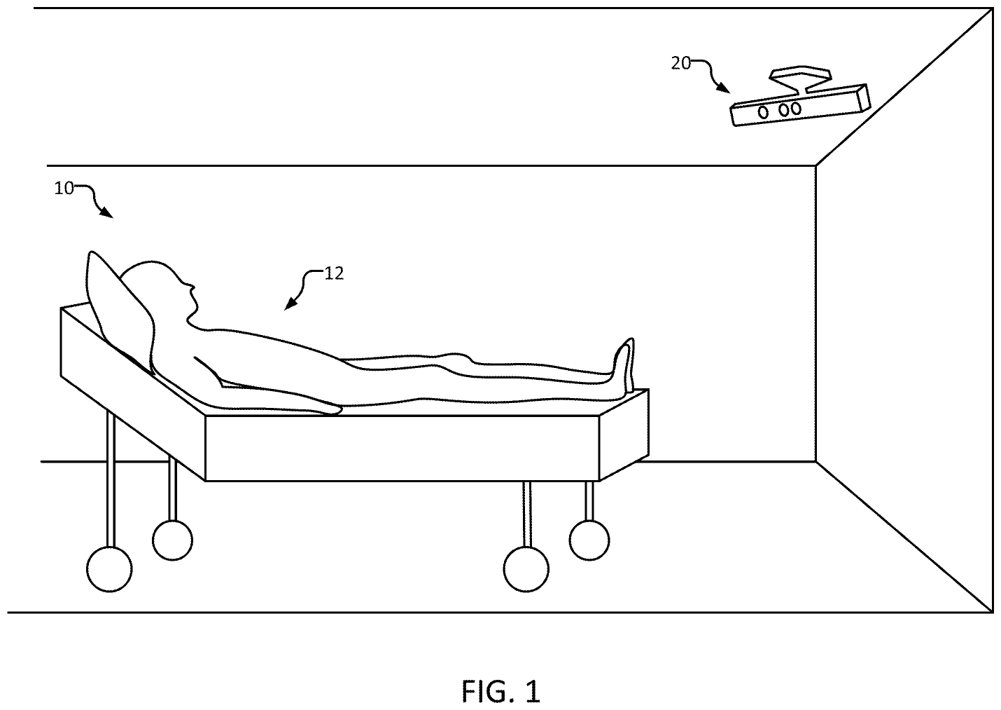

Referring to FIG. 1, a room 10 can include a patient 12 and a monitoring device 20. Monitoring device 20 may be positioned in a variety of locations (e.g., room 10), including, but not limited to, hospital rooms including intensive care unit, step down units, and standard hospital beds, in nursing homes, assisted living, or in private homes. In some cases, monitoring device 20 can be tailored for use in the elderly at home or for infants in NICUs, hospital beds, or at home as an apnea monitor to prevent sudden infant death, or to identify infants or children left in cars to avoid “hot-car-deaths.” In some cases, monitoring device 20 can be mounted on a ceiling of room 10. In some cases, the monitoring device 20 can be mounted perpendicular to a location of a chest of patient 12. In some cases, monitoring device 20 can be mounted to see a face of patient 12. In some cases, monitoring device 20 can identify patient 12 in room 10. In some cases, monitoring device 20 can include motors and or telephoto lenses to provide movement of monitoring device and improved resolution 20. In these cases, once monitoring device 20 identifies patient 12, monitoring device 20 can track patient 12 if patient 12 moves about room 10.

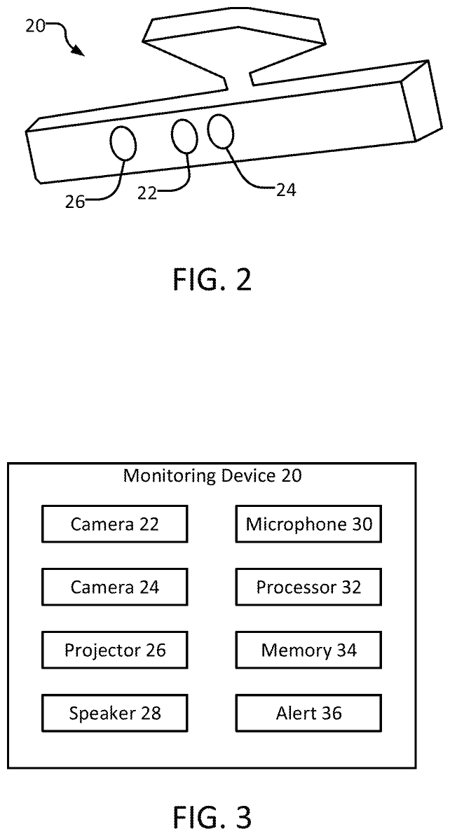

Referring to FIGS. 2 and 3, monitoring device 20 can include a first camera 22, a second camera 24, a projector 26, a speaker 28, a microphone 30, a processor 32, a memory 34, and an alert 36.

First camera 22 can be an RGB (Red Green Blue) video camera that can record visible light. First camera can also be an RGB video camera array with two cameras providing stereoscopic depth information. Data gathered from camera 22 can be broken into multiple parameters, such as:
AC_{R} Alternating signal from video image in red color.
DC_{R} Constant signal or minimal signal from video image in red color.
AC_{G} Alternating signal from video image in green color.
DC_{G} Constant signal or minimal signal from video image in green color.
AC_{B} Alternating signal from video image in blue color.
DC_{B} Constant signal or minimal signal from video image in blue color.

The data collected by first camera 22 can be used to determine a plurality of patient parameters described herein.

Second camera 24 can be an infrared (IR) camera that can record infrared light and can work in the dark. Projector 26 can be an infrared projector that projects a pattern in infrared light onto the scene (e.g., room 10). The infrared camera can be calibrated to the pattern and can detect distortion of the pattern by objects in the field of view. Distortions in the IR pattern can be used to calculate a depth image which can give 3-dimensional data of the field of view to a depth resolution of 1 mm. The monitoring device 20 can identify “joints” (e.g., 20 joints) in the patient (e.g., head, neck, shoulders, elbows, hands, hips, knees, ankles, feet, etc.). These “joints” may not be the actual skeletal joint but rather an estimate of joint location by analysis of patient shape. The monitoring device can track thoracic movement based on both joint location (shoulders, neck, head, spine) as well as thoracic position. The depth image can have a high resolution (1 mm) in z-axis (depth) and can detect thoracic displacement. Data gathered from camera 24 and projector 26 can be broken into multiple parameters, such as:
AC_{I} Alternating signal from video image in infrared color.
DC_{I} Constant signal or minimal signal from video image in infrared color.
AC_{O} Alternating signal from video image in other color such as ultraviolet or other choices.
DC_{O} Constant signal or minimal signal from video image in other color such as ultraviolet or other choices.

The data collected by the second camera 24 and the projector 26 can be used to determine a plurality of patient parameters described herein.

The AC and DC signals from the video image (e.g., first camera 22, second camera 24, and/or projector 26) may be single pixels, or averages of pixel fields, or optimized choices of pixels, such as all pixels that meet a certain threshold for quality or consistency.

The data collected from first camera 22, second camera 24, and/or projector 26 can be analyzed to determine oxygen saturation, heart rate, respiratory rate, respiratory motion and adequacy of ventilation, cardiac perfusion, distribution of blood flow, blood pressure, and a plurality of other parameters, as described below. In some cases, analysis of video images can provide continuous or intermittent assessment of multiple parameters of patient health.

In some cases, pulsatility of the heart beat can be identified in exposed skin, and calculation of the oxygen saturation of the blood (SaO2) can be achieved by calculating the ratio of the pulsatile signal intensity (AC) by the non-pulsatile signal intensity (DC) for different frequencies of light. In some cases, ratios include AC_{RED}/DC_{RED}/AC_{Infrared}/DC_{Infrared}. The comparison of the AC/DC signals of any two frequencies can be used (Red/Infrared, Blue/Infrared, Green/Infrared, Red/Blue, Red/Green, Blue/Green), as described below. If other frequencies are available, the ratios can be calculated and used.

The monitoring device 20 can calculate ratios for a combination of colors of light, as outlined below.
R_{RI}=ln(AC_{R}/DC_{R})/ln(AC_{I}/DC_{I}),
R_{GI}=ln(AC_{G}/DC_{G})/ln(AC_{I}/DC_{I}),
R_{BI}=ln(AC_{B}/DC_{B})/ln(AC_{I}/DC_{I}),
R_{RG}=ln(AC_{R}/DC_{R})/ln(AC_{G}/DC_{G}),
R_{RB}=ln(AC_{R}/DC_{R})/ln(AC_{B}/DC_{B}),
R_{GB}=ln(AC_{G}/DC_{G})/ln(AC_{B}/DC_{B}),
R_{λ1λ2}=ln(AC_{λ1}/DC_{λ1})/ln(AC_{λ2}/DC_{λ2}),
   where ln is the natural log λ1 is a frequency of a first light signal and λ2 is a frequency of a second light signal. In some cases, if other colors are available from first camera 22, second camera 24, projector 26, and/or other available cameras. The ratios above can be used to derive oxygen saturation (SaO₂). In some cases, the following ratios can be used to derive oxygen saturation.
R_{RI}=(AC_{R}/DC_{R})/(AC_{I}/DC₁)
R_{GI}=(AC_{G}/DC_{G})/(AC_{I}/DC_{I})
R_{BI}=(AC_{B}/DC_{B})/(AC_{I}/DC_{I})
R_{RG}=(AC_{R}/DC_{R})/(AC_{G}/DC_{G})
R_{RB}=(AC_{R}/DC_{R})/(AC_{B}/DC_{B})
R_{GB}=(AC_{G}/DC_{G})/(AC_{B}/DC_{B})
R_{λ1λ2}=(AC_{λ1}/DC_{λ1})/(AC_{λ2}/DC_{λ2})

In some cases, oxygen saturation can be measured using the following equation,

SaO₂=m_{λ1λ2}*R_{λ1λ2}+b_{λ1λ2}

where m_{λ1λ2} is the slope and b_{λ1λ2} is the intercept.

In some cases, oxygen saturation can be derived from a multiparameter algorithm derived from a linear or non-linear combination of measurements from different frequencies of light. In some cases, oxygen saturation can be derived using the following equation,

SaO₂=m_{RI}*R_{RI}+m_{GI}*R_{GI}+m_{BI}*R_{BI}+m_{RG}*R_{RG}+m_{RB}*R_{RB}+m_{GM}*R_{GB}+m_{λ1λ2}*R_{λ1λ2}+b,

where m_{λ1λ2} is the slope and b_{λ1λ2} is the intercept.

In some cases, m_{xy} is a parameter fitting the R_{xy} to experimentally derived data and b is an intercept for that data. In some cases, non-linear parameters (m_{xy}) may be used to fit experimental data. In some cases, non-linear or interacting parameters (m_{xy}) may be used to fit the experimental data. In some cases, ratios (m_{xy}*R_{xy}/R_{QT} or m_{xy}*R_{xy}*R_{QT}) may be used to fit the data.

In some cases, measurement of background light levels either with or without a color standard in the image can be used to adjust parameters (m & b) to correct for variations in light intensity of color spectrum of room lighting. The m and b parameters can be set by experimental calibration. Data from recordings from experimental subjects who are breathing normoxic and hypoxic mixtures of gas to achieve various oxygen saturation levels can be used to set individual m and b parameters. Once set, the m and b parameters can be fixed for the device unless dynamic variations are needed based on measurement of light intensity or color spectrum measurements.

In some cases, signals derived from video camera(s) (e.g., first camera 22, second camera 24, and/or projector 26) can be used to determine heart rate (HR) measurements. Variations in color intensity over time, (red, green, blue, infrared, or other colors depending on the camera) from areas of skin such as the face, forehead, chest, or other areas, can be used to measure heart rate. In some cases, heart rate can be derived from filtering the image followed by Fast Fourier Transform (FFT) or from peak detection or from other algorithms.

In some cases, signals derived from the video camera(s) (e.g., first camera 22, second camera 24, and/or projector 26) can be used to determine respiratory rate (RR) measurements. Respiration or chronic breathing is important for the maintenance of life and interruption of breathing can lead to major adverse events such as death. Medications such as opiates can suppress respiration and may lead to apnea (the cessation of breathing) or respiratory depression or respiratory arrest. The signal can be from red, green, blue, infrared, or 3D imaging systems. Respiratory rate can be derived from variations in intensity of red, green, blue, infrared signal intensities or a combination of those signals over time. In some cases, heart rate can be derived from filtering the image followed by Fast Fourier Transform (FFT), from peak detection or from other algorithms. In some cases, respiratory rate can be derived from variations in motion of the chest or thorax, or abdomen from a three-dimensional camera. In some cases, a combination and/or comparison of respiratory rates measured separately from the first camera 22, second camera 24 and/or projector 36 can improve accuracy of the respiratory rate calculation and reduce false alarms for the detection of apnea.

Analysis of changes in the signal intensity of skin can be used to identify and measure heart rate. Specifically, analysis of image intensity of signals from the skin in red, green, blue, infrared, or other colors can identify changes caused by pulsation of the heart. These changes with pulsations of the heart can be seen in any exposed skin and therefore, tracking the face can allow concentration on exposed skin such as the forehead, cheeks, or ears. Any exposed skin can be used for these measurements using R_{GB} cameras. Skin covered by clothing or blankets can be used for these measurements using the IR camera.

In some cases, cardiac perfusion or adequacy of blood flow can be derived from variations in the perfusion of skin. Perfusion can be measured by identification of pulsatility of the skin caused by blood flow with each heartbeat. For example, blood flow to the face (e.g., nose, forehead, cheeks) can vary in comparison to blood flow of lateral skin locations due to variations in the veins present. Accordingly, variations in the distribution of blood flow or the uniformity of blood flow, detected by variations in the pulsatility of the video signal can provide information on adequacy of perfusion.

In some cases, perfusion or distribution of blood flow can be assessed in a free-flap created for repair of a surgical defect. The perfusion or distribution can be assessed by observing the pulsatility of blood flow signal with a video camera (e.g., first camera 22, second camera 24, and/or projector 26). After surgical repair using a flap of tissue based on a vascular supply, commonly known as a free flap, occlusion or clotting of the blood supply can lead to ischemia of the vascular flap and flap failure. Early recognition of compromise of the blood supply can prevent flap ischemia and ultimately failure of the surgical repair. Video images (e.g., from first camera 22, second camera 24, and/or projector 26) can be used to derive cardiac perfusion in a free flap. A variation in the distribution of pulsatile signals across the flap, loss of pulsatility, or desaturation of the flap can be used was a warning of impending free flap failure and the need for intervention.

In some cases, blood pressure can be measured by pulse shape analysis detected remotely by IR or R_{GB} camera after calibration to a known blood pressure. Blood pressure can be monitored over time in patients remotely by detecting pulse shape and or wave reflections in the IR or R_{GB} signals.

In some cases, composite variables can be derived from combinations of parameters such as heart rate, respiratory rate, saturation, temperature, perfusion, mobility, responsiveness, etc. In some cases, these composite variables can predict impending respiratory or cardiac events. A dimensionless parameter or composite variable can be derived from linear and/or non-linear combinations of other parameters and associated with impending cardiac and respiratory events. In some cases, the composite variable can be calculated and displayed to inform staff that patient's status should be reviewed. In some cases, an impending respiratory or cardiac event can be detected up to 12 hours before the event would occur.

In some cases, monitoring device 20 can monitor motion of the chest, face, and/or abdomen from first camera 22, second camera 24, and/or projector 26. In some cases, monitoring device 20 can identify respiratory motion that is not coordinated, implying obstructed breathing. Machine learning techniques or explicit algorithms looking at coordinated motion of the head, face, and or body can identify obstructed breathing patterns of motion, such as uncoordinated motion, uneven shoulders, asymmetry in motion, etc. In some cases, monitoring device 20 can identify obstructed breathing when the chest rises with inspiration but no sound of respiration is identified from microphone 30.

In some cases, monitoring device 20 can monitor motion of the chest, face, and/or abdomen from first camera 22, second camera 24, and/or projector 26 to identify vomiting. Machine learning techniques or explicit algorithms looking at coordinated motion of the head, face, and or body can be used to identify motion associated with vomiting, such as hunching over. In some cases, monitoring device 20 can identify vomiting motion of the body and/or a retching sound identified from microphone 30. In some cases, monitoring device 20 can identify vomiting from a retching sound identified from microphone 30.

In some cases, monitoring device 20 can monitor the patient using first camera 22, second camera 24, and/or projector 26 to identify the location of the patient in the bed. If it is not safe for a patient to get out of bed without supervision, the monitoring device 20 can identify where the patient is relative to the sides of the bed. In some cases, parameters can be set to generate alert 36 if the patient moves from the approved location (e.g., a center of the bed, a distance from the edge of the bed, etc.) providing an “out of bed” patient alert. Alarms can be set to notify clinical staff (nursing) when a patient attempts to get out of bed. The out of bed alarm can be used to reduce falls in hospitalized patients, especially because patient falls are a major source of morbidity and mortality and cost in hospitalized patients.

In some cases, monitoring device 20 can monitor the patient's face, arms, and legs using first camera 22, second camera 24, and/or projector 26. In some cases, when a patient develops a new facial asymmetry, the patient can be identified as needing evaluation for possible stroke. In patients that have facial asymmetry, predominant motion on one side, one arm, or one leg, or who fail to move one side of their body, the monitoring device 20 can identify the patient as needing evaluation for possible stroke. Initial patient motion can be used to either manually or automatically set parameters for baseline mobility for assessment of stroke. If a patient has motion or other asymmetries at initial evaluation, because of pre-existing body asymmetries or prior history of stroke, the baseline motion or asymmetries can be quantified to allow identification of new or changes in mobility or symmetry of motion indicating possible acute stroke.

Speaker 28 can allow the monitoring device to communicate with a patient. Microphone 30 can allow the patient to communicate with the monitoring device 20. In some cases, microphone 30 can be an array of microphones (e.g., four microphones) that can provide cancellation of sound and can locate sound sources. The monitoring device 20 driver software can identify the patient as separate from the background or other patients.

Processor 32 can be general or special purpose microprocessors or both, or any other kind of central processing unit with or without a graphical processing unit (GPU). Generally, a central processing unit will receive instructions and data from a read-only memory or a random access memory or both (e.g., memory 34). The essential elements of a computer can include a central processing unit for performing or executing instructions and one or more memory devices for storing instructions and data. Generally, processor 32 can be operatively coupled to receive data from or transfer data to, or both, one or more mass storage devices for storing data, e.g., magnetic, magneto-optical disks, or optical disks.

Memory 34 can include computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor 32 and the memory 34 can be supplemented by, or incorporated in, special purpose logic circuitry. Processor 32 and memory 34 can be used to implement the monitoring, detection and alerting systems and procedures described herein.

Alert 36 can generate an alert based on detected data. As described above, monitoring system 20 can calculate a respiratory rate. The monitoring system 22 can use infrared cameras (e.g., first camera 22, second camera 24, and/or projector 26) and can work in the dark or indoor lighting, so as to not disturb patients. In some cases, the monitoring device 20 can track patient respirations optically. If respirations cannot be detected, the monitoring device 20 can enter an alert mode 36. In some cases, upon entering alert mode 36, the monitoring device 20 can begin storing video recordings for future analysis. In some cases, the alert 36 is a combination of alarm states that is used to reduce false alarm rates.

When the respiratory rate is greater than a predetermined threshold, the monitoring device 20 may not generate an alert. However, when the respiratory rate is below a predetermined threshold, or when the respiratory rate is not detected, the alert 36 may determine the patient has a possible apnea. In some cases, when the respiratory rate is below a predetermined threshold, or when the respiratory rate is not detected, the alert 36 can determine if movement of the patient is detected. If movement is detected, the alert 36 can reset and monitoring device 20 can attempt to detect respirations. In some cases, if no patient movement is detected, the alert 36 can move to another step of determining if a patient is experiencing an apnea. In some cases, the monitoring device 20 can use speaker 28 to request that the patient move, and determine if the patient has moved. In some cases, speaker 28 can produce an audible statement, such as “Mr. or Mrs. Patient, please wave your hand,” (e.g., head, hand, foot, etc.). In some cases, the patient's preferred salutation can be entered into the monitoring device 20 in advance. In some cases, if patient movement is detected, alert 36 can reset, such as if the patient responds by waving their hand, then the monitoring device 20 can reset the alert 36 and attempt to detect respirations. If no patient movement has been detected by monitoring device 20, monitoring device 20 can use speaker 28 to request that the patient speak. In some cases, speaker 28 can produce an audible statement, such as “Mr. or Mrs. Patient, are you alright?” In some cases, monitoring device 20 can have voice recognition with voice localization, such that only speech from the monitored patient will be sufficient to reset the alert 36 based on speech. If speech is detected from the monitored patient, the alert 36 can be reset and attempt to detect respirations. Speech detection can help reduce false alarms because if the patient is speaking, the patient is breathing, and if the patient is breathing, an alert for detection of apnea is likely a false alarm. If speech is not detected from the monitored patient, alert 36 can generate an alert indicating an apnea has been detected. Therefore, if patient movement, or respirations, waving a hand, or speech, cannot be detected, then the monitoring device 20 can enter an alert state and can text message the nurse and/or physician or anesthesiologist on call. In some cases, the sequence or combination of patient parameters checked and identified can reduce false alarm rates and provide reasonable sensitivity and specificity.

In some cases, the monitoring device 20 can be used to detect and prevent respiratory arrests in hospitalized patients. In some cases, the alert 36 can identify when a patient is getting out of bed without assistance, as some patients may fall out of bed or attempt to get out of bed on their own, which can lead to a fall or other accident. Falls can cause significant patient injury with fractured hips, and hip fracture has a 20% mortality rate. Therefore, hospitals spend enormous effort to reduce patient falls. As such, monitoring device 20 can be set to identify when patients attempt to get out of bed and the nurse, or other personnel can be notified.

In some cases, monitoring device 20 can recognize speaker independent verbal commands such as “HELP” or “NURSE” and alert 36 can call the nurse to the patient bed side. In some cases, speech recognition can be built into the monitoring device 20 and can be programmed to signal nursing staff or other commands.

In some cases, alert 36 can trigger video observation of patient. In some cases, the monitoring device 20 can be networked to hospital monitoring system to provide the video observation. In some cases, alert 36 can trigger based on a sequence of patient monitoring or voice recognition can be used to initiate video surveillance or video chatting with the patient from a central nurses' station.

In some cases, when alert 36 is triggered (e.g., speech, motion, etc.), monitoring device 20 can be used to control devices in the patient's room, such as the TV, computer, lights, etc. In some cases, alert 36 can be an audio alarm, electronic signal to nursing station, electronic signal to monitoring system, text message to nursing or physicians or other staff, or combination of alarms.

In some cases, composite variables can be used to detect impending respiratory and cardiac arrest. Multi-parameter, composite variables which combine heart rate, respiratory rate, oxygen saturation, perfusion score, and or mobility score can provide a dimensionless parameter that indicates the probability or likelihood of a cardiac or respiratory arrest. A composite parameter can be displayed that indicates the likelihood of clinical deterioration based on a combination of measured parameters (heart rate, respiratory rate, oxygen saturation, perfusion, etc). In some cases, if the composite variable crosses a threshold, alert 36 can be generated.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

What is claimed is:

1. A method of monitoring a patient, the method comprising:
collecting a video image of the patient, wherein the video image comprises at least two colors;
deriving at least two signals from each of the at least two colors of the video image, wherein the at least two signals comprise an alternating signal, a constant signal, or a frequency of a signal;
calculating a ratio of the at least two signals from each of the at least two colors; and
determining a saturation of oxygen of the patient based on the ratio.

2. A method of monitoring a patient, the method comprising:
collecting a video image of the patient, wherein the video image comprises at least two colors;
deriving at least two signals from each of the at least two colors of the video image;
determining a saturation of oxygen of the patient based on the at least two signals.

3. The method of claim 2, further comprising calculating a ratio of the at least two signals from each of the at least two colors, wherein the saturation of oxygen of the patient is based on the ratio.

4. The method of claim 2, wherein the ratio comprises at least one of an alternating current (AC) signal of the at least two signals or a direct current (DC) signal of the at least two signals, and wherein the AC signal and the DC signal are derived from one or more colors of light, the one or more colors of light comprising a red light (R), a green light (G), a blue light (B), an infrared light, an ultraviolet light, or a light wavelength (λ).

5. The method of claim 4, wherein the ratio comprises one or more of:
R_{RI}=ln(AC_{R}/DC_{R})/ln(AC_{I}/DC_{I})
R_{GI}=ln(AC_{G}/DC_{G})/ln(AC_{I}/DC_{I})
R_{BI}=ln(AC_{B}/DC_{B})/ln(AC_{I}/DC_{I})
R_{RG}=ln(AC_{R}/DC_{R})/ln(AC_{G}/DC_{G})
R_{RB}=ln(AC_{R}/DC_{R})/ln(AC_{B}/DC_{B})
R_{GB}=ln(AC_{G}/DC_{G})/ln(AC_{B}/DC_{B})
R_{λ1λ2}=ln(AC_{λ1}/DC_{λ1})/ln(AC_{λ2}/DC_{λ2}).

6. The method of claim 4, wherein the ratio comprises one or more of:
R_{RI}=(AC_{R}/DC_{R})/(AC_{I}/DC_{I})
R_{GI}=(AC_{G}/DC_{G})/(AC_{I}/DC_{I})
R_{BI}=(AC_{B}/DC_{B})/(AC_{I}/DC_{I})
R_{RG}=(AC_{R}/DC_{R})/(AC_{G}/DC_{G})
R_{RB}=(AC_{R}/DC_{R})/(AC_{B}/DC_{B})
R_{GB}=(AC_{G}/DC_{G})/(AC_{B}/DC_{B})
R_{λ1λ2}=(AC_{λ1}/DC_{λ1})/(AC_{λ2}/DC_{λ2})

7. The method of claim 2, wherein the saturation of oxygen of the patient is determined by a linear equation or a non-linear equation.

8. The method of claim 7, wherein the saturation of oxygen of the patient is determined using multiple parameters, wherein the multiple parameters comprise various frequencies of light from the video image.

9. The method of claim 7, wherein parameters of the linear equation or non-linear equation are modified based on measurements of a background light level to correct for variations in a light intensity of color spectrum or a room lighting.

10. The method of claim 2, further comprising measuring a heart rate of the patient using the video image, wherein measuring the heart rate comprises detecting a variation in intensity of one of the at least two colors, wherein the variation in intensity is detected from at least one of a cheek, a forehead, or a chest of the patient.

11. The method of claim 10, wherein measuring the heart rate further comprises filtering the at least two signals from each of the at least two colors using at least one of a fast fourier transform or a peak detection.

12. The method of claim 2, further comprising measuring a respiratory rate of the patient using the video image, wherein measuring the respiratory rate comprises detecting a variation in motion of the patient, wherein the variation in motion is detected from at least one of a chest, a thorax, or an abdomen of the patient.

13. The method of claim 2, further comprising measuring a perfusion of the patient using the video image, wherein measuring the perfusion comprises measuring a pulsatility of a skin of the patient, and detecting a variation in at least one of a distribution of blood flow or a uniformity of blood flow of the pulsatility.

14. The method of claim 13, wherein the skin of the patient is a free-flap created for repair of a surgical defect.

15. The method of claim 2, further comprising:
detecting a respiration rate or a heart rate of the patient using the video image;
when the respiration rate or the heart rate is not detected, requesting the patient initiate a movement of a body part;
detecting the movement of the body part of the patient using the video image;
when the movement of the body part is not detected, requesting the patient make a sound;
detecting the sound using a microphone;
when the sound is not detected, triggering an alarm.

16. The method of claim 15, wherein the alarm is at least one of an audio alarm, an electronic signal to nursing station, an electronic signal to monitoring system, a text message to another person.

17. The method of claim 2, further comprising deriving a composite variable, wherein the composite variable comprises one or more parameters collected using the video image, wherein the one or more parameters are a heart rate, a respiratory rate, the saturation of oxygen, a temperature, a perfusion, a mobility, or a responsiveness.

18. The method of claim 17, wherein the composite variable is derived from at least one of a linear combination or a non-linear combination.

19. The method of claim 17, further comprising predicting at least one of a respiratory event or a cardiac event using the composite variable.

20. The method of claim 2, further comprising detecting an obstructed breathing pattern of the patient using the video image, wherein detecting the obstructed breathing pattern of the patient comprises monitoring a breathing motion of the patient, and detecting an uncoordinated breathing motion.

21. The method of claim 20, wherein detecting the obstructed breathing pattern further comprises monitoring a breathing sound of the patient.

22. The method of claim 2, further comprising detecting a vomiting motion of the patient using the video image, wherein detecting the vomiting motion of the patient comprises monitoring a motion of the patient, and detecting the motion is indicative of vomiting.

23. The method of claim 22, wherein detecting the vomiting motion further comprises monitoring a sound of the patient.

24. The method of claim 2, further comprising monitoring a location of the patient in a bed using the video image, and triggering an alarm when the patient moves outside of a designated location of the bed.

25. The method of claim 2, further comprising monitoring an asymmetry of the patient using the video image, wherein monitoring the asymmetry comprises identifying at least one of a facial asymmetry, a lack of movement on a side of a body of the patient, or a predominant motion on one side of the body, wherein the predominate motion comprises at least one of an arm or a leg.

26. The method of claim 2, further compromising monitoring of the blood pressure of the patient using the video image and analyzing the pulsatile signal from at least one color, wherein the shape of the waveform, after calibration, will provide a measurement of the blood pressure.
